(19) United States
(12) Patent Application Publication
(10) Pub. No.: US 2025/0300037 A1
(43) Pub. Date: Sep. 25, 2025

(54) THERMAL DISSIPATION STRUCTURES FOR ULTRASOUND PROBES

(71) Applicant: BFLY OPERATIONS, INC., Burlington, MA (US)

(72) Inventors: Jason Fischman, Burlington, MA (US); Matthew R. Hageman, Burlington, MA (US); Timothy A. Hyde, Columbia, SC (US)

(21) Appl. No.: 18/862,288

(22) PCT Filed: May 9, 2023
(86) PCT No.: PCT/US2023/021491

§ 371 Date: Nov. 1, 2024

Related U.S. Application Data
(60) Provisional application No. 63/339,900, filed on May 9, 2022.

(51) Int. Cl. H01L 23/373; A61B 8/00; H01L 23/498

(52) U.S. Cl. CPC H01L 23/3737; A61B 8/4427; A61B 8/4494; A61B 8/546; A61B 8/56; H01L 23/49827

(57) ABSTRACT

The present disclosure provides an ultrasound device including thermal dissipation features. The ultrasound device is an ultrasound probe in some situations and includes thermal dissipation features allowing for increased runtime at higher power consumption rates. The thermal dissipation features include an interposer with thermal vias, a heat spreader, a heat sink, a single piece probe housing, and a chassis, or various combinations of such features.

### CROSS REFERENCE TO RELATED APPLICATIONS

This application claims the benefit of and priority to U.S. Provisional Patent Application Ser. No. 63/339,900 filed May 9, 2022, the entire contents of which is incorporated by reference herein.

### BACKGROUND

### Field

The present disclosure relates to thermal dissipation structures for ultrasound devices such as ultrasound probes, and more specifically to thermal vias that conduct heat away from ultrasonic transducers.

### Related Art

Some ultrasound apparatuses take the form of ultrasound probes. Some such ultrasound probes include ultrasonic transducers and associated circuitry that produce ultrasonic signals, and also in turn produce heat.

### BRIEF SUMMARY

According to an aspect of the present disclosure, an apparatus is provided, comprising: a handheld ultrasound probe weighing between 2,500 grams and 100 grams, or less than 500 grams, having a length of less than 300 mm, and being couplable to a smartphone or tablet. The handheld ultrasound probe contains: a handheld housing; a rear cap coupled to the handheld housing; a shroud coupled to the handheld housing; and a lens coupled to the shroud, wherein the handheld housing, rear cap, shroud, and lens are coupled to define an enclosed space. The handheld ultrasound probe further comprises a semiconductor chip or chip stack disposed behind the lens within the enclosed space and comprising an array of microscale ultrasonic transducers and integrated circuitry. The handheld ultrasound probe further comprises an interposer disposed behind the semiconductor chip or chip stack within the enclosed space; and at least one circuit board disposed behind the interposer within the enclosed space and electrically coupled to the interposer. The interposer comprises a plurality of epoxy-filled copper-plated thermal vias having inner diameters between 5 mil and 15 mil, spaced from each other at a pitch between 10 mil and 30 mil (e.g., between 12 mil and 20 mil, or any other value within the range from 10 mil to 30 mil), and covering in combination between 5% and 15% of an area of one side of the interposer. The handheld ultrasound probe is configured to operate in a runtime mode with a power consumption of at least 5 Watts for at least 15 minutes.

According to an aspect of the present disclosure, a method of operating an ultrasound apparatus is provided, comprising: with a semiconductor chip or chip stack comprising an array of microscale ultrasonic transducers and integrated circuitry, transmitting and receiving ultrasound signals through a lens of the ultrasound apparatus; and dissipating heat generated by the semiconductor chip or chip stack through a plurality of epoxy-filled copper-plated thermal vias disposed in an interposer coupled to a back of the semiconductor chip or chip stack. The plurality of epoxy-filled copper-plated thermal vias cover in combination between 5% and 15% of an area of one side of the interposer.

According to an aspect of the present disclosure, an ultrasound imaging apparatus is provided, comprising: a semiconductor chip or chip stack comprising an array of microscale ultrasonic transducers and integrated circuitry; and an interposer coupled to the semiconductor chip or chip stack and comprising a plurality of dedicated thermal vias covering between 5% and 15% of an area of a side of the interposer and configured to dissipate heat generated by the semiconductor chip or chip stack.

### BRIEF DESCRIPTION OF DRAWINGS

The following brief description of the drawings is meant to assist the understanding of one skilled in the art and is not meant to unduly limit any present or future claims relating to the present disclosure. Various aspects and embodiments are described with reference to the following figures. It should be appreciated by one skilled in the art that the figures are not necessarily drawn to scale. Items appearing in multiple figures are indicated by the same reference number in all the figures in which they appear.

FIG. 1 illustrates an ultrasound device according to embodiments of the present disclosure.

FIG. 2 illustrates a cross-sectional view of the ultrasound device of FIG. 1 taken along the length L.

FIG. 3 illustrates an expanded view of a portion of the ultrasound device shown in FIG. 2.

FIGS. 4A and 4B illustrate dimensions for thermal vias that may be used as the thermal vias of FIG. 3.

FIG. 5A illustrates a portion of an ultrasound device having an interposer with a hole formed in it to accommodate a portion of a shroud adapter, according to embodiments of the present disclosure.

FIG. 5B illustrates a perspective view of the interposer shown in FIG. 5A.

FIG. 6 illustrates a thermal dissipation structure for an ultrasound device, including a heat sink, according to embodiments of the present disclosure.

FIG. 7 illustrates in schematic form an ultrasound-on-chip device as may be implemented in ultrasound devices of the types described herein, according to embodiments of the present disclosure.

FIGS. 8A and 8B illustrate an ultrasound device of the types described herein configured to communicate with an external processing device.

### DETAILED DESCRIPTION

The following detailed description is meant to help the understanding of one skilled in the art, and is not meant in any way to unduly limit present or future claims related to the present disclosure.

Ultrasonic imaging devices project ultrasonic signals. For medical applications, such ultrasonic signals are projected into a patient's body. The ultrasonic imaging devices can be used to detect the reverberations or reflections of those signals, digitize such, and through various data processing and manipulation techniques, create images and data depicting certain features of the patient's body.

One type of ultrasonic imaging device uses an ultrasound-on-chip device, which is a microscale chip or chip stack having integrated ultrasonic transducers and circuitry. The chip may be a semiconductor chip, such as a silicon chip. The ultrasonic transducers may be microscale ultrasonic transducers arranged in an array. The integrated circuitry may include analog and/or digital circuitry for controlling operation of the ultrasonic transducers and/or processing signals produced by the ultrasonic transducers. In some embodiments, the integrated circuitry includes analog to digital signal converters and beamformers. According to an embodiment of the present disclosure the ultrasonic transducers and the aforementioned associated circuitry are integrated into a single silicon chip.

One type of ultrasonic transducer that can be integrated into the single silicon chip or a chip stack is a capacitive micromachined ultrasonic transducer (CMUT). A CMUT may provide a wide range of ultrasonic signals, for example from 0.5 Mhz to 12 Mhz, thus allowing for scanning of different parts of the human body with the single CMUT based chip or chip stack. However, operating the CMUTs tends to generate a significant amount of heat—for instance, the circuitry associated with the CMUTs can generate a significant amount of heat during operation—that can be disadvantageous in certain ways. One way is the temperature experienced by the patient. If the ultrasonic imaging device becomes too hot, it can cause discomfort or harm to the patient. Similarly, the operator holding the ultrasonic imaging device can be harmed if the device becomes too hot. Also, the circuitry of the ultrasonic imaging device can be negatively impacted by elevated heat and temperatures. For instance, if the ultrasonic imaging device becomes too hot the internal circuitry may be unable to maintain stable operation. If the ultrasonic imaging device becomes too hot, the endurance of the circuitry may also be negatively impacted.

With that, the present disclosure includes a number of combinations of embodied features relating to conduction of heat generated by CMUT transducers away from the CMUT transducers and the ultrasound-on-chip device, thereby addressing the issues noted herein.

Aspects of the present disclosure provide an ultrasound device having a CMUT based ultrasonic transducer as part of an ultrasound-on-chip device, thermal dissipation features configured to enhance thermal dissipation from the ultrasound-on-chip device, allowing for adequate operating power and runtime of the ultrasound device. The ultrasound device may be a point of care ultrasound device, such as a handheld ultrasound probe, a patch, or other wearable configurations. In operation, the circuitry and transducers consume power and generate heat. The ultrasound device may further include additional application specific integrated circuits (ASICs), circuit boards, Field Programmable Gate Arrays (FPGAs), or other circuitry that consumes power and generates heat. As described herein, the power consumption and resulting heat generation can limit runtime since the ultrasound device may heat up, risking danger to the operator or subject, and may negatively impact operation of the device. The thermal dissipation features described herein may enhance thermal dissipation, thus reducing heat build-up and allowing the ultrasound device to operate for a longer time at a given power consumption level.

The thermal dissipation features include an interposer with thermal dissipation vias. The interposer may be a printed circuit board (PCB) in some or all of the embodiments described herein. The interposer may be positioned adjacent and in contact with the ultrasound-on-chip device and/or between the ultrasound-on-chip device and a heat spreader or heat sink. The thermal vias may conduct heat from a backside of the ultrasound-on-chip device to the heat spreader and/or heat sink, thus reducing heat buildup at the ultrasound-on-chip device. The thermal vias are in some embodiments positioned to cover an area ranging from 5-15% of the total surface area of one side of the interposer, therefore providing meaningful thermal dissipation while leaving sufficient space on the surface of the interposer for electrical signal wiring to accommodate the electrical connections to the circuitry and ultrasonic transducers of the ultrasound-on-chip device. For instance, the thermal vias may cover an area which, as a result of the presence of the thermal vias, has an effective thermal conductivity between 15-50 W/m-K. The effective thermal conductivity is a measure of thermal dissipation and may be measured in units of W/m-K or an equivalent unit. The thermal vias are part of a thermal via assembly including a metal sheet on the surface of the interposer that connects the thermal vias to one another. The metal sheet covers an area ranging from 10-20% of the total surface area of one side of the interposer.

An ultrasound device may include a chassis serving as a thermal mass and configured to support circuitry of the ultrasound device. The chassis may be disposed within a housing of the ultrasound device and arranged along a length of the housing. The chassis may be formed of a material suitable for operating as a thermal mass. Additionally, the chassis may support electronics, for example in the form of a circuit board or discrete electronic components.

The thermal dissipation features of an ultrasound device may include a thermally conductive single piece housing. In some embodiments, the single piece housing is a unibody housing. The thermally conductive single piece housing may lack a thermal interface along its length in some embodiments, thus facilitating substantially continuous thermal flow from the heat source to the remainder of the housing. Such substantially continuous thermal flow facilitates heat dissipation and avoids the generation of hot spots on the ultrasound device. That, in turn, lengthens runtime of the ultrasound device for a given operating power level.

An ultrasound device may have a combination of the thermal features described above. Two or more such thermal features may be used in combination to increase the thermal dissipation of the ultrasound device and therefore to increase the runtime of the ultrasound device for a given power consumption level. In some embodiments, an ultrasound probe includes all of the thermal dissipation features described above. In some embodiments only one is used.

The aspects and embodiments described above, as well as additional aspects and embodiments, are described further below. These aspects and/or embodiments may be used individually, all together, or in any combination of two or more, as the disclosure is not limited in this respect.

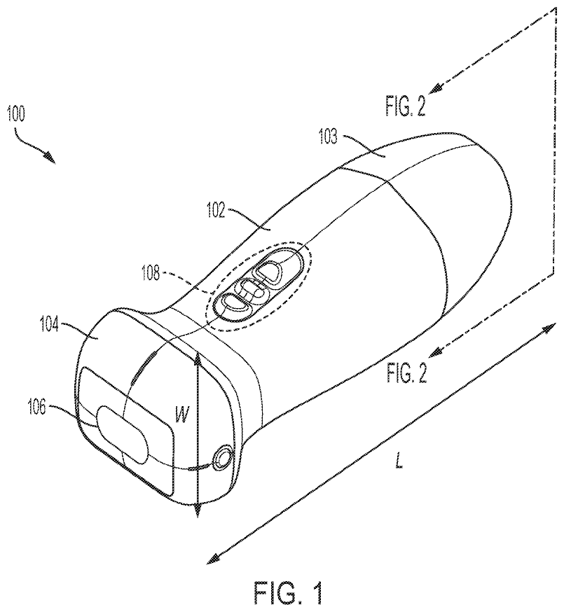

FIG. 1 illustrates an ultrasound device according to aspects of the present disclosure. The ultrasound device may be a handheld ultrasound probe for use in point of care ultrasound (POCUS) applications. The ultrasound device 100 includes a housing 102, a rear cap 103, a shroud 104 and a lens 106. Buttons 108 are positioned on the housing 102.

The housing 102 is configured to house internal electronics of the ultrasound device 100. The housing 102, shroud 104, and lens 106 may be coupled together to define an enclosed space in which electronics may be disposed. For instance, an ultrasound-on-chip device may be disposed within the enclosed space defined by the housing 102, shroud 104, and lens 106. Additional circuitry in the form of integrated circuits on dies (e.g., ASICs), FPGAs, and/or circuit boards may also be disposed within the space defined by the housing 102, shroud 104, and lens 106. In some embodiments, for assembly the electronics may slide into the housing 102.

The housing 102 may be made of a thermally conductive material having a thermal conductivity coefficient of at least 50 (W m−1 K−1) at 20° C. and 1 bar. For example, the housing 102 may be metal, such as aluminum. Other conductive materials, including other metallic materials, may be used for the housing 102 in various embodiments. The thermally conductive nature of the housing 102 facilitates thermal dissipation. Heat generated by the electronics of the ultrasound device may be more readily spread through the housing 102 due to its thermally conductive nature than if the housing 102 were made of a generally non-thermally-conductive material having a thermal conductivity coefficient of less than 50 (W m−1 K−1) at 20° C. and 1 bar. Such spreading of the heat may facilitate dissipation of the heat and provide for longer runtime of the ultrasound device 100. The housing 102 may also serve as a thermal mass, further contributing to the handling of heat generated by the internal circuitry of the ultrasound device 100.

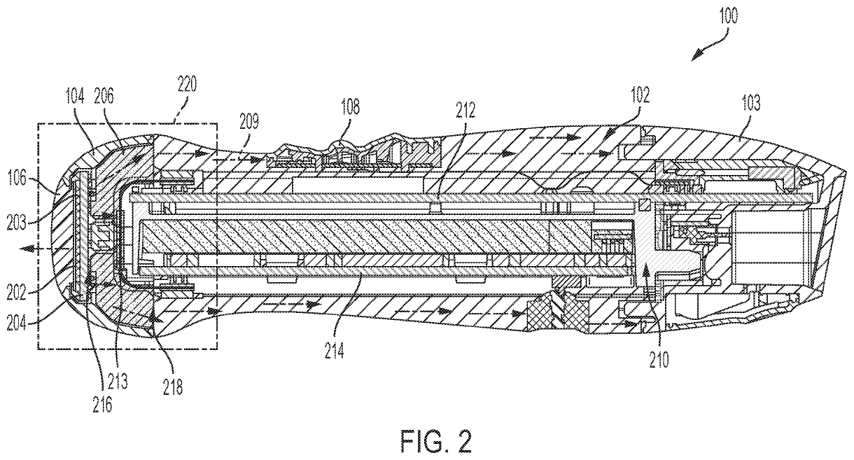

In some embodiments, the housing 102 has a single piece construction, meaning that the housing is a single piece of material. The single piece construction may provide a substantially continuous thermal flow path, thus enhancing heat dissipation by avoiding undue heat build-up at thermal interfaces. The single piece construction of the housing 102 is illustrated in connection with FIG. 2 and described further in connection with that figure.

The housing 102 can be constructed from aluminum and can be made from a single piece of aluminum. There are various thermal benefits to the housing being constructed from a single piece of aluminum by way of machining, such as continuity of material cross section and uniform material properties. The housing 102 can form an elongated inner cavity that defines therein an internal longitudinal axis extending along the inner cavity. The housing 102 can surround the longitudinal axis and the cavity radially, except for an access opening in the housing corresponding to the buttons 108.

The rear cap 103 is positioned at the rear of the ultrasound device 100. The rear cap 103 is at the opposite end of the ultrasound device from the lens 106. In some embodiments, the rear cap 103 includes a receptacle for receiving a communication cable for plugging the ultrasound device into an external processing device, such as a smartphone, tablet computer, or laptop computer. The rear cap 103 may be a separable piece from the housing 102, or can be a fully integrated part of the housing 102, wherein the rear cap 103 and the housing 102 are a unified part of single piece construction.

The rear cap 103 may be made of various materials. In some embodiments, the rear cap 103 is thermally conductive. For instance, the rear cap 103 may be made of the same material as the housing 102 in some embodiments, including any of the materials described above for the housing 102. Thus, in some embodiments the rear cap 103 may be made of aluminum. In other embodiments, the rear cap 103 may be made of a different material than the housing 102. In some embodiments, the rear cap 103 is made of plastic.

The shroud 104 is configured to couple the lens 106 to the housing 102 in a secure manner. The shroud 104 may also function in combination with the housing 102, rear cap 103, and lens 106 to define an enclosed space in which the electronics of the ultrasound device 100 are disposed.

The shroud 104 may be formed of any suitable material. In some embodiments, the shroud 104 is formed of a thermally insulating material, having a thermal conductivity coefficient of less than 50 (W m−1 K−1) at 20° C. and 1 bar. The shroud may be configured to contact a subject during ultrasound imaging. The insulating nature of the shroud 104 may prevent discomfort or harm to the subject that could otherwise result if the shroud were to heat up significantly during operation of the ultrasound device 100. The shroud 104 may be made of materials such as plastic, polymer, or metals with a thermal conductivity coefficient of less than 50 (W m−1 K−1) at 20° C. and 1 bar. The shroud 104 and the lens 106 can together form a structure that has an outside surface with a convex cross-sectional shape, that has a cylindrical curvature. The curvature can be constant or can vary radially.

The lens 106 is configured to pass ultrasound signals. Ultrasound signals generated by the ultrasound device 100 and transmitted outwardly, as well as ultrasound signals received by the ultrasound device 100 may pass through the lens 106. As described above, the lens in combination with the shroud 104, housing 102, and rear cap 103 may define an enclosed space in which the electronics of the ultrasound device 100 are disposed. The ultrasound-on-chip device can be located immediately adjacent to the lens 106. The lens 106 may be formed of any material suitable for operating as a lens for ultrasound signals. For example, the lens may be formed of rubber, room temperature vulcanizing (RTV) silicone, or an elastomer such as Pebax® available from Arkema of King of Prussia, Pennsylvania, and may be homogenous in construction or may include particles dispersed in a solid. The lens 106 may be thermally insulating, conducting no heat or conducting a negligible amount of heat compared to the housing 102. Thus, the lens 106 may generally be less thermally conductive than the housing 102.

The buttons 108 may control operation of the ultrasound device 100. For example, the buttons 108 may control functions such as depth of imaging, gain, image capture, switching between modes, color on/off, wireless pairing with an external processing device, activation of a battery status indicator, and/or soft resetting of the ultrasound device. Alternative or additional functions may be controlled as well.

The buttons 108 may be provided to control the desired function of the ultrasound device 100. In the example shown, three buttons 108 are included. The three buttons 108 are located adjacent to one another and are sequential in a straight line, thereby providing positive ergonomic interactions for a user. It is advantageous to operate the functions of the ultrasound device with one button or set of buttons 108 at a single location. The three buttons 108 can be covered by a single unified flexible cover, such as a rubber cover.

The ultrasound device 100 may be sized and constructed to be portable, such as being handheld. The ultrasound device has a length L and a width W. The length L may be 100 mm-300 mm (e.g., 140 mm, 175 mm, or other value) including any value or range of values within that range. The width W may be 20 mm-100 m including any value or range of values within that range. The ultrasound device 100 may have a weight of 200 grams-500 grams (e.g., 265 g, 312 g, or other value). The ultrasound device 100 may have a length of 140 mm and a weight of 265 g.

FIG. 2 illustrates a cross-sectional view of the ultrasound device 100 taken along the length L as shown in FIG. 1. As shown in FIG. 2, the ultrasound device 100 includes an ultrasound-on-chip device 202, heat spreader 203, an interposer 204, a shroud adapter 206, a chassis 210, circuit board 212 and circuit board 214. The interposer includes thermal vias 216 which contact the shroud adapter 206 over an area representing a thermal interface. Thermally conductive grease may be positioned at the area of contact between the interposer 204 and the shroud adapter 206. A second thermal interface 218 is formed at the intersection of the shroud adapter 206 and the housing 102.

The ultrasound-on-chip device 202 includes integrated ultrasonic transducers and circuitry integrated on a common substrate, such as a silicon substrate or other semiconductor substrate. The ultrasonic transducers and circuitry in combination may operate to transmit and receive ultrasound signals. For example, the ultrasound transducers may emit ultrasound signals through the lens 106 and may receive ultrasound signals through the lens 106.

The ultrasonic transducers of the ultrasound-on-chip device 202 may be one of various types of ultrasonic transducers. They may be capacitive micromachined ultrasonic transducers (CMUTs), piezoelectric micromachined ultrasonic transducers (PMUTs), or other suitable ultrasonic transducers, including other types of microfabricated ultrasonic transducers. CMUTs may require higher driving voltages than PMUTs, which in turn may result in higher power consumption of the ultrasound device. As described further below, power consumption leads to heat generation which can negatively impact runtime of the ultrasound device.

The ultrasonic transducers of the ultrasound-on-chip device 202 may form an array. In some embodiments, the ultrasonic transducers form a 2D array, although in alternative embodiments the ultrasonic transducers may form a 1.5D array or a 1D array. The array includes hundreds or thousands of ultrasonic transducers in some embodiments. For example, the ultrasound-on-chip device in some embodiments includes an array of between 7,000 and 12,000 (e.g., 9,000) ultrasonic transducers arranged in a 2D array. Other numbers of ultrasonic transducers may be implemented in alternative embodiments.

The circuitry of the ultrasound-on-chip device 202 may be analog and/or digital circuitry suitable for controlling operation of the ultrasonic transducers to transmit and receive ultrasound signals. For example, analog circuitry such as pulser circuits, time gain compensation circuits, transimpedance amplifiers (TIAs), and filters may be disposed on the ultrasound-on-chip device. Analog to digital converters may also be included to convert received analog signals to digital signals. Digital circuitry such as filters, summing circuitry, or digital amplification circuitry may also be included, among other possible types of digital circuitry on the ultrasound-on-chip device.

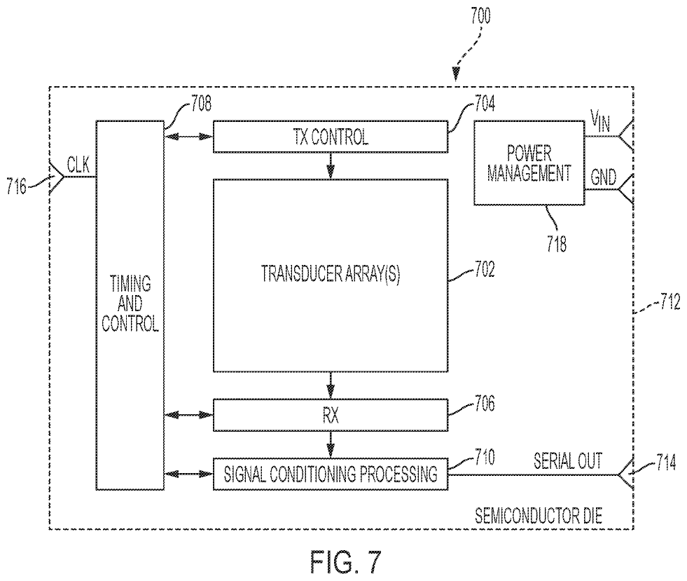

A non-limiting example of suitable ultrasound-on-chip devices that may be implemented as the ultrasound-on-chip device 202 are described in U.S. patent application Ser. No. 14/635,197 filed Mar. 2, 2015 and issued as U.S. Pat. No. 9,067,779; U.S. patent application Ser. No. 15/626,711 filed Jun. 19, 2017 and published as U.S. Patent Pub. No. 2017/0360399 A1, each of which applications is owned by the Assignee of the present application and each of which is incorporated by reference herein in its entirety. Moreover, further details of a potential implementation of the ultrasound-on-chip device 202 are described below in connection FIG. 7.

The ultrasound-on-chip device 202 represents a source of heat generation within the ultrasound device 100. In operation, the circuitry of the ultrasound-on-chip device 202 consumes power, resulting in heat. The amount of heat generated depends on the rate of power consumption and the runtime of the ultrasound device 100. The longer the runtime for a given rate of power consumption, the more heat is generated.

The heat spreader 203 is configured to spread heat generated by the ultrasound-on-chip device 202. In this example, the heat spreader 203 is coupled to the back of the ultrasound-on-chip device 202. The heat spreader 203 is formed of a thermally conductive material so that heat generated by the ultrasound-on-chip device 202 may be spread throughout the heat spreader 203. It should be appreciated that the heat spreader 203 is an optional feature in the construction of FIG. 2.

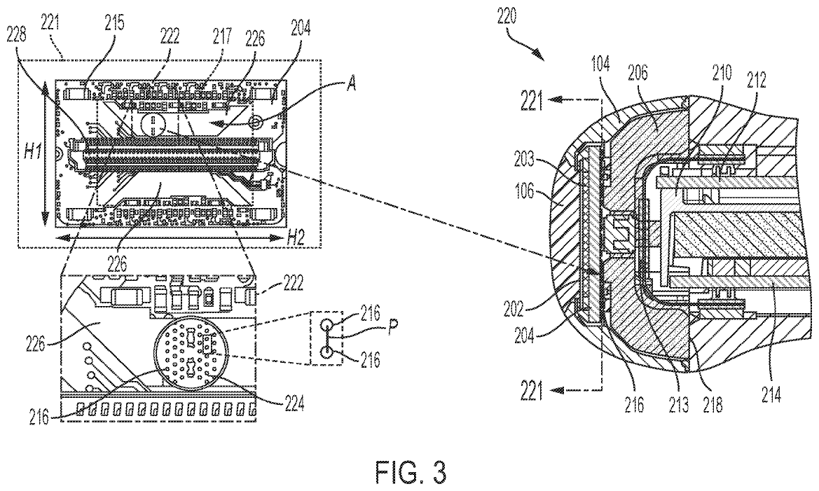

The interposer 204, which may be a printed circuit board (PCB), performs multiple functions. One function performed by the interposer 204 is to route electrical signals between the circuitry of the ultrasound-on-chip device 202 and additional circuitry of the ultrasound device 100. For example, the ultrasound-on-chip device 202 may communicate with the circuit board 212 and/or circuit board 214. Additionally, a circuit board 213 may be provided to process signals or route signals between the various circuits of the ultrasound device 100. For that reason, the interposer 204 is positioned electrically between the ultrasound-on-chip device 202 and the circuit boards 212, 214, and 213. A second function performed by the interposer 204 is to provide a thermal pathway between the ultrasound-on-chip device 202 and the shroud adapter 206. For that reason, the interposer 204 includes thermal vias. The thermal vias 216 contact the shroud adapter 206 at an area representing a thermal interface. The thermal vias 216 are positioned to conduct heat from the back of the ultrasound-on-chip device 202 to the shroud adapter 206 to facilitate dissipation of such heat from the ultrasound-on-chip device. As described further below in connection with FIG. 3, some embodiments include a thermally conductive (e.g., metal) sheet covering part of a surface of one or both sides of the interposer 204 and connecting the thermal vias 216 to one another. The combination of the thermal vias and the conductive sheet represents a thermal via assembly. In at least some embodiments, the thermal vias are dedicated thermal vias in that they are provided for the singular purpose of providing a thermal pathway. In at least some embodiments, the thermal vias are not configured as part of an electrical signal path. When the thermal vias are dedicated thermal vias, they are not used to conduct electrical signals through the interposer or from the interposer to any other component of the ultrasound device. In some embodiments, the thermal vias are connected to a system electrical ground. The interposer 204 and thermal vias 216 are described further below in connection with FIG. 3.

The shroud adapter 206 is configured to support the shroud 104 and to provide a thermally conductive pathway between the interposer 204 and the housing 102. The shroud adapter 206 operates as a heat spreader and/or a heat sink. The shroud adapter 206 may have any suitable shape for performing those functions. The shroud adapter may be formed of a thermally conductive material. In some embodiments, the shroud adapter has a thermal conductivity coefficient of at least 50 (W m−1 K−1) at 20° C. and 1 bar. In some embodiments the shroud adapter 206 is formed of the same material as the housing 102. In some embodiments, the shroud adapter is formed of metal, such as aluminum.

The shroud adapter 206 may be configured to interface with the housing 102 as shown. The shroud adapter 206 and housing 102 meet at a thermal interface 218. The larger the area of the thermal interface, the greater the heat transfer that takes place between the shroud adapter 206 and the housing 102.

The chassis 210 may serve multiple functions. One function performed by the chassis 210 is to support other components of the ultrasound device 100. For example, as shown, the circuit boards 212 and 214 may be mounted to the chassis 210. The circuit board 212 is mounted to a first side of the chassis 210 and the circuit board 214 is mounted to a second, opposite side of the chassis 210. The chassis 210 has a length that is at least 30% of L in some embodiments, at least 50% of L, or at least 75% of L, including any values within those ranges. A second function performed by the chassis 210 can be to operate as a thermal mass and/or a heat spreader. The chassis 210 may be formed of any suitable material to operate as a thermal mass. In operating as a thermal mass, the chassis 210 may absorb and store heat, such as heat generated by the ultrasound-on-chip device 202, the circuit board 212, and/or the circuit board 214. Such heat absorption and storage by the chassis 210 may further increase the runtime of the ultrasound device 100 before overheating occurs.

The circuit boards 212 and 214 may include suitable circuitry for carrying out some of the functions of the ultrasound device 100. For example, processing circuitry such as beamforming, image formation, compression, power management, mode control, and other functions may be performed by circuitry connected to one or both of the circuit boards 212 and 214. In some embodiments, only one of the circuit boards 212 and 214 may be included within the ultrasound device 100.

The circuit boards 212 and 214 contain circuitry that consumes power and represents a heat source. The positioning of the chassis 210 adjacent the circuit boards 212 and 214 facilitates the chassis' absorption and storage of some of the heat generated by the circuitry of those circuit boards. The positioning of the housing 102 adjacent the circuit boards 212 and 214 serves to provide a thermal pathway to dissipate heat generated by the circuitry of those circuit boards. Thus, positioning the circuit boards 212 and 214 between both the chassis 210 and the housing 102 serves to sink and dissipate heat generate by the circuitry of those circuit boards.

In operation of the ultrasound device 100, ultrasound signals are generated by the ultrasound-on-chip device 202 and transmitted outwardly through the lens 106 and/or ultrasound signals pass through the lens 106 and are received by the ultrasound-on-chip device 202. Heat is generated by the ultrasound-on-chip device 202, the circuit board 212, the circuit board 214, and the circuit board 213. As shown, the generated heat may travel in the manner indicated by arrows 209. That is, heat generated by the ultrasound-on-chip device 202 may pass through the thermal vias 216 of the interposer 204 to the shroud adapter 206. From there, the generated heat passes to the housing 102. As described above in connection with FIG. 1, the housing 102 has a single piece construction in at least some embodiments. As a result, heat may travel freely through the housing 102, without encountering problematic interfaces. Heat passing from the shroud adapter 206 to the housing 102 at the thermal interface 218 may travel rearward in the housing and dissipate. As described above in connection with the circuit boards 212 and 214, heat generated by the circuitry of those circuit boards may be sunk by the chassis 210 operating as a thermal mass and may dissipate by conducting along the housing 102 to the surface area of the housing 102. Thus, heat generated by the circuitry of the ultrasound device 100 may be dissipated through much of the ultrasound device itself, allowing for higher power operation and longer runtimes as described further below.

The interposer 204 and thermal vias 216 are now described in greater detail with respect to FIG. 3, which illustrates an expanded view of a portion 220 of the ultrasound device 100. Several of the components illustrated are the same as in FIG. 2, and thus those components are not described in detail again here. The interposer 204 is illustrated in FIG. 3 both in the portion 220 and in the call-out 221, which is a view of the interposer 204 from inside the ultrasound device 100 looking toward the lens 106 as indicated by the dashed arrows. The interposer 204 may be a PCB. As shown in the call-out 221, the interposer 204 includes circuitry 215, a conductive sheet region 226, and the thermal vias 216.

The conductive sheet region 226 is thermally conductive, is positioned on the surface of one side of the interposer 204 as shown, and connects thermal vias 216 to one another. In at least some embodiments, the opposite side of the interposer 204 also includes a conductive sheet region, and that conductive sheet region may be identical to the conductive sheet region 226 or substantially the same in at least one of material, size, and positioning. The conductive sheet regions on the opposing sides of the interposer may be aligned with each other. In some embodiments, the conductive sheet region 226 has a thermal conductivity coefficient of at least 50 (W m−1 K−1) at 20° C. and 1 bar. In some embodiments, the conductive sheet region is formed of metal, such as copper. The conductive sheet region 226 is a copper pour region in some embodiments. The conductive sheet region may be formed of a different material than the housing 102 in some embodiments. The conductive sheet region 226 connects thermal vias 216 to one another. A portion of the conductive sheet region 226 with underlying thermal vias 216 is further illustrated in the call-out 222. In the call-out 222, a portion of the conductive sheet of the conductive sheet region 226 is removed for an area 224 to reveal the individual thermal vias 216. In practice, the individual thermal vias 216 would not be visible from the perspective of call-out 222 because the conductive sheet region 226 would extend across the area 224. The combination of the thermal vias 216 and the conductive sheet region 226 connecting the thermal vias 216 to one another represents a thermal via assembly.

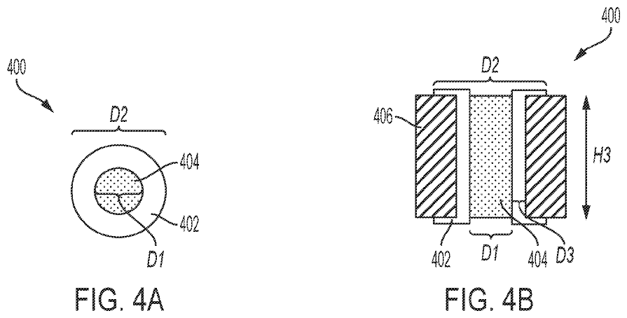

The thermal vias 216 are positioned and sized to perform meaningful thermal dissipation while not interfering with the electrical functionality of the interposer 204. For instance, the thermal vias may provide an effective thermal conductivity of between 15-50 W/m-K for the area in which they are disposed. As described previously, the interposer electrically interfaces between the ultrasound-on-chip device 202 and additional circuitry of the ultrasound device 100, such as the circuitry of circuit boards 212, 213, and 214. The number of electrical connections may be significant, for instance one hundred or more. For example, the illustrated pin connector 228 includes 160 pins. In other embodiments, a pin connector with between 100 and 200 pins may be included. The circuitry of the ultrasound-on-chip device 202 may include approximately 100 nets (e.g., between 80 and 120 nets, including any value in that range). The greater the area of the interposer 204 dedicated to thermal dissipation structures, the more complicated will be the electrical routing. In some embodiments, the combined surface area of thermal vias at one side of the interposer—which referring to the embodiment of FIGS. 4A and 4B described below is given by combined surface area of thermal vias=(number of thermal vias)×π(D2/2)²—is between 5% and 15% of the total surface area (H1× H2 in FIG. 3) of that side of the interposer 204. The surface area A of the conductive sheet region 226 is greater than the combined surface area of the thermal vias at that side of the interposer since the conductive sheet region extends across the thermal vias 216 and covers the spaces between the thermal vias 216. In some embodiments, the surface area of the conductive sheet region 226 is between 10% and 20% of the surface area of the side of the interposer 204 on which the conductive sheet is disposed. The remaining surface area of the interposer 204, not covered by the conductive sheet region 226, may be used for electrical routing. In some embodiments, the surface area (H1×H2) of one side of the interposer 204 is between 700 mm² and 1,000 mm². In some embodiments, the thermal vias 216 are positioned across an area corresponding to the surface area A of the conductive sheet region 226, which may be between 60 mm²-150 mm² of the surface area of the one side of the interposer 204, or any value or range of values within that range (e.g., 120 mm², 130 mm², 140 mm², or between 80 mm² and 150 mm²). In some embodiments, due to the spacing between individual thermal vias 216, their combined surface area at the one side of the interposer is not equal to the surface area A of the conductive sheet region 226, but instead is less. For example, in some embodiments the total surface area of the thermal vias 216 at the one side of the interposer may be between 60 mm²-130 mm², for example approximately 80 mm², 90 mm², or any other value within that range. For instance, in some embodiments, the area (H1× H2) of one side of the interposer 204 is approximately 840 mm², the interposer includes thermal vias having a combined surface area at the one side of the interposer 204 of approximately 90 mm², and the thermal vias are positioned across an area of approximately 130 mm² corresponding to the area A of the conductive sheet region 226 at the one side of the interposer 204. The described areas occupied by the thermal vias and conductive sheet apply equally to the opposite side of the interposer. Therefore, in at least some embodiments, the described thermal vias and conductive sheets occupy the listed areas of both sides of the interposer.

As shown, the conductive sheet region 226 and the thermal vias 216 may be positioned relatively centrally with respect to the interposer 204. For example, they may be positioned within an inner region 217 of the interposer in FIG. 3. In this non-limiting example, the thermal vias 216 are adjacent the pin connector 228—positioned on two sides of the pin connector—and much of the circuitry 215 is located closer to a periphery of the interposer 204, outside the inner region 217. That is, in some embodiments, an interposer of an ultrasound device comprises a centrally located pin connector, thermal vias adjacent either side of the pin connector within an inner region of the interposer, and circuitry adjacent the thermal vias. Such a configuration facilitates the dedication of a meaningful amount of the interposer area for the placement of the thermal vias.

As also shown in FIG. 3, the thermal vias 216 may be grouped together, rather than being evenly distributed across the interposer 204. In FIG. 3, one thermal via assembly comprising a conductive sheet region 226 and corresponding thermal vias 216 is on one side of the pin connector 228, and a second thermal via assembly comprising a conductive sheet region 226 and corresponding thermal vias 216 is on the other side of the pin connector 228.

The thermal vias may be formed of various thermally conductive materials. For example, they may comprise a copper outer-formed for example by copper plating- and a conductive core. In some embodiments, the conductive core comprises a conductive epoxy. In other embodiments, the conductive core comprises copper, which in some embodiments may be in the form of a copper paste. Other materials may be used in alternative embodiments. In some embodiments, the thermal vias comprise a thermally conductive outer and a non-thermally conductive core.

In some embodiments, tens or hundreds of thermal vias 216 are provided as part of the interposer of the ultrasound device. For example, between 100-600 thermal vias may be provided, including any number within that range. In some embodiments, between 400-500 thermal vias may be provided, or between 450-500 thermal vias.

FIGS. 4A and 4B illustrate non-limiting examples of suitable dimensions for thermal vias that may be used as the thermal vias 216. FIG. 4A is a top view of a thermal via of the type illustrated in FIG. 3. The thermal via 400 includes an outer portion 402 filled with an inner conductive core 404. The inner conductive core 404 has a first, smaller diameter D1 while the outer portion 402 has a larger diameter D2. D1 may be between 5 mil and 15 mil, such as being approximately 8 mil, or any other value within that range. D2 may be between 5 mil and 13 mil larger than D1 as an example. The thickness D3 of the thermal via walls may be a result of the manufacturing techniques used to form the thermal vias. For instance, if plating is used, the thickness D3 may be is the plating thickness. The thickness D3 may be between 15 microns and 50 microns in some embodiments. The height H3 (visible in FIG. 4B, which is a side view) may be substantially equal to the thickness of the interposer 406. For example, H3 may be between 50 microns and 2,000 microns in some embodiments.

The thermal vias may be spaced by a pitch P shown in FIG. 3. The pitch P may be kept small to allow for the thermal vias to take up a sufficiently small space on the interposer to leave sufficient room for the electrical wiring, as well as being manufacturable. In some embodiments, the pitch P may be between 10 mil (where a mil equals one-thousandth of an inch) and 25 mil, between 10 mil and 20 mil, or any value or range of values within those ranges. For example, the pitch may be 16 mil.

Combinations of the thermal via inner diameters, materials, and pitch, and the resulting effective thermal conductivity, are shown in Table 1.

TABLE 1

| Via Inner | Thickness of | Fill | Via | Effective Thermal | |
|---|---|---|---|---|---|
| Diameter | Copper Plating | Material | Pitch | Conductivity | |
| 6 mil | N/A | Cu | 18 mil | 23 | W/m-K |
| 12 mil | 50 um Cu | Epoxy | 22 mil | 35 | W/m-K |
| 6.8 mil | 15 um | Cu Paste | 18 mil | 13 | W/m-K |
| 8 mil | 20 um | Cu Paste | 18 mil | 17.5 | W/m-K |
| 8 mil | 20 um | Epoxy | 16 mil | 19.5 | W/m-K |

As shown, effective thermal conductivities between 13 and 50 W/m-K are provided by the enumerated combinations of thermal via inner diameters, materials, and pitch. The effective thermal conductivity is that of an area including thermal vias having the enumerated characteristics. The total amount of heat dissipated will therefore depend on the size of the area including such thermal vias.

Various combinations of the sizing, spacing, number, and material of thermal vias may be utilized in connection with aspects of the present disclosure. According to one embodiment, an ultrasound device of the type illustrated in FIGS. 1 and 2 is provided. The surface area (H1×H2) of one side of the interposer is approximately 840 mm², and the interposer comprises approximately 470 thermal vias. The thermal vias have a combined surface area at the one side of the interposer of approximately 95 mm² and are positioned across approximately 135 mm² of the surface area of the one side of the interposer, corresponding to the area A of a conductive sheet region. The vias may be copper-plated with a conductive epoxy core. In some alternative embodiments, the vias have a non-conductive core. The vias may have inner diameters D1 of approximately 8 mil and a copper plating thickness of approximately 20 microns. The same areas apply to the opposite side of the interposer in at least some embodiments. Therefore, in some embodiments, the thermal vias have the listed combined surface area on both sides of the interposer.

The thermal dissipation features illustrated in connection with FIGS. 1-4 increase the runtime of the ultrasound device 100. The circuitry of the ultrasound device 100 consumes power and therefore generates heat. Often, increased ultrasound imaging performance is associated with higher levels of power consumption, because generation of stronger ultrasound signals and faster operation of the circuitry is typically associated with greater levels of power consumption. Yet, the greater the power consumption, the greater the amount of heat generated. Such heat generation can limit the continuous amount of time the ultrasound device can operate safely. If the ultrasound device heats up too much, patient and operator safety may be compromised. For example, the heat may cause patient and/or operator discomfort, or the patient and/or operator may be burned. Alternatively, or additionally, the circuitry of the ultrasound device may be damaged or malfunction due to overheating. By dissipating the heat generated by the circuitry of the ultrasound device, the thermal dissipation structures described herein serve to extend the runtime of the ultrasound device and allow for higher power operation. For instance, the thermal dissipation structures described herein may maintain the temperature of those portions of the ultrasonic imaging device which contact a patient (e.g., shroud 104 and lens 106) below 43° C. That temperature is the upper permitted temperature limit for some applications of the ultrasonic imaging device, such as certain healthcare applications. The ultrasonic imaging device may be maintained below such temperature for the power consumption levels and runtimes described herein.

In some embodiments, one or more of the thermal dissipation features described in connection with FIGS. 1-4, or the subsequent figures of this disclosure, may facilitate longer ultrasound device runtimes at higher power consumption levels than would otherwise be achievable. For example, the ultrasound devices of the types described herein, including the thermal dissipation structures of the types described herein, may operate at power levels of at least 3 Watts, at least 4 Watts, at least 5 Watts, between 3 and 10 Watts, or any value within that range. The runtime at such power levels may be at least 10 minutes, at least 15 minutes, at least 20 minutes, or between 15 and 30 minutes, as examples. For instance, in some embodiments the ultrasound device may operate at a power level of approximately 5.5 Watts for at least 20 minutes before overheating, which may represent nearly a doubling in the runtime compared to what would be achievable without the combination of thermal dissipation features described.

The impact of the surface area of the interposer 204 over which the thermal vias 216 are disposed on the ultrasound device runtime may be significant. For example, for a given ultrasound device 100 and a given power consumption level, the inclusion of the thermal vias 216 may add at least a 50% increase in runtime, or more. As a non-limiting example, for a simulated ultrasound device having no thermal vias in the interposer, the runtime may be approximately 12 minutes. Including approximately 10 mm² of thermal vias 216 may increase the runtime to approximately 14 minutes. Including approximately 25 mm² of thermal vias 216 in the interposer 204 may increase the runtime to approximately 16 minutes. Including approximately 50 mm² of thermal vias 216 in the interposer 204 increases the runtime to approximately 18 minutes. Including approximately 75 mm² of thermal vias 216 in the interposer 204 may increase the runtime beyond 18 minutes. Thus, the heat dissipation by the thermal vias 216 of the interposer 204 may meaningfully extend the runtime of the ultrasound device.

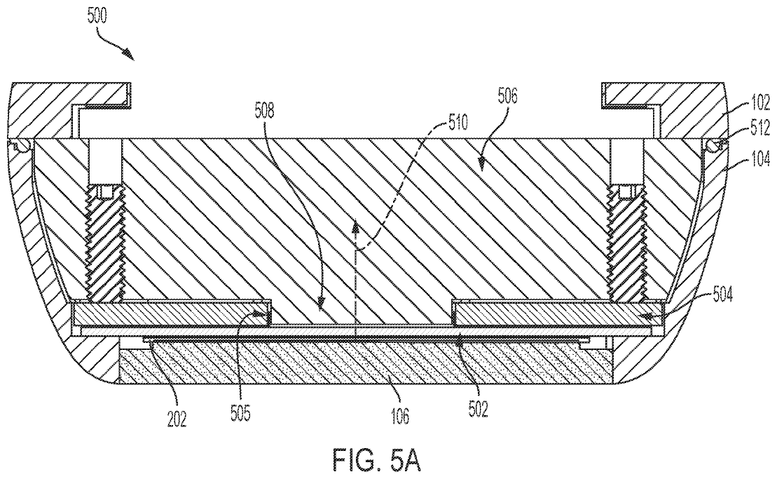

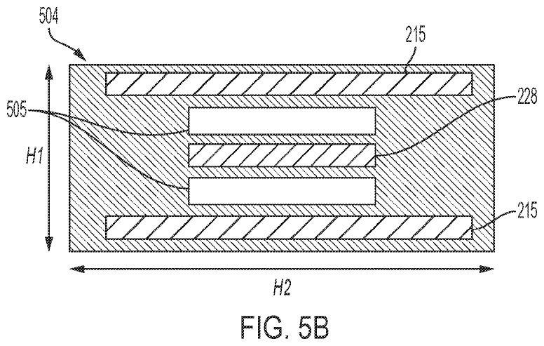

Alternative thermal dissipation structures compared to those illustrated in FIGS. 2-4 are provided in alternative embodiments. According to an aspect of the present disclosure, an ultrasound device has an interposer disposed partially between an ultrasound-on-chip device or a heat spreader coupled to the ultrasound-on-chip device and a thermally conductive shroud adapter, wherein the interposer comprises at least one opening through which the shroud adapter contacts the ultrasound-on-chip device or the heat spreader. For example, and referring back to FIG. 2, the illustrated interposer 204 may alternatively lack the thermal vias 216 and instead have an opening through which the shroud adapter 206 contacts the back of the ultrasound-on-chip device 202 or a heat spreader included in the ultrasound device. In this manner, a thermal dissipation path is provided from the ultrasound-on-chip device 202 to the thermally conductive shroud adapter 206 to conduct heat back behind the ultrasound-on-chip device 202. FIG. 5A illustrates an example.

The illustrated portion 500 of an ultrasound device includes the ultrasound-on-chip device 202, a heat spreader 502, interposer 504, and shroud adapter 506. A seal ring 512, such as an O-ring, may be provided to seal the connection between the housing 102 and the shroud 104. The interposer 504 includes an opening 505 through which a protrusion 508 of the shroud adapter 506 passes, so that the shroud adapter 506 contacts the heat spreader 502.

The heat spreader 502 is configured to spread heat generated by the ultrasound-on-chip device 202. In this example, the heat spreader 502 is coupled to the back of the ultrasound-on-chip device 202. The heat spreader 502 is formed of a thermally conductive material so that heat generated by the ultrasound-on-chip device 202 may be spread throughout the heat spreader 502. It should be appreciated that the heat spreader 502 is an optional feature in the construction of FIG. 5A.

The interposer 504 may provide electrical connectivity between the ultrasound-on-chip device 202 and additional circuitry of the ultrasound device, such additional circuitry being omitted from FIG. 5A for simplicity of illustration. The additional circuitry may be the same as in FIG. 2. The opening 505 of the interposer 504 may be sized such that the surface area of the interposer is sufficient to accommodate the electrical wiring for connecting the ultrasound-on-chip device 202 with the additional circuitry of the ultrasound device. For example, the opening 505 may replace the conductive sheet region 226 and thermal vias 216 shown in FIG. 3. FIG. 5B illustrates a perspective view of the interposer 504, showing the pin connector 228 and circuitry 215 in simplified block diagram form. In this view, the interposer 504 includes two openings 505 corresponding substantially to the placement of conductive sheet regions 226 in the interposer 204 of FIG. 3. One opening 505 is on one side of the pin connector 228 and the other opening 505 is on the other side of the pin connector 228. The total in-plane area A₅₀₅ of the two openings 505 may be between 5% to 15% of the surface area (H1×H2) of one side of the interposer, or from 10% to 20% of the surface area of the one side of the interposer in some embodiments. The opening 505 may be positioned at a suitable location of the interposer 504 to allow for the remaining area of the interposer to accommodate the pin connector 228, circuitry 215, and electrical wiring.

The protrusion 508 of the shroud adapter 506 may be sized to fit within the opening 505 of the interposer 504. In some embodiments, the protrusion 508 is sized to substantially fill the opening 505 of the interposer 504. The location of the protrusion 508 on the shroud adapter 506 is selected to align with the opening 505 in the interposer 504.

The thermal dissipation structures of FIG. 5A may conduct heat away from the ultrasound-on-chip device 202. For instance, as shown in FIG. 5A, the heat spreader 502 and shroud adapter 506 serve to draw heat away from the ultrasound-on-chip device 202 in the direction of the arrow 510.

An ultrasound device including the construction shown in FIG. 5A may include the additional thermal dissipation features illustrated in FIG. 2. For example, a housing with a single piece construction and a chassis supporting a circuit board may be provided.

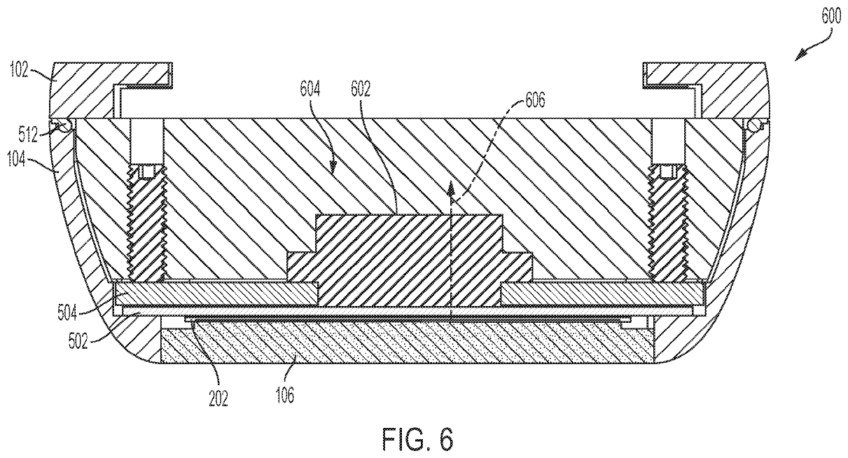

FIG. 6 illustrates another alternative construction of a thermal dissipation structure for an ultrasound device, including a heat sink. The portion 600 of the ultrasound device includes many of the same components described previously in connection with the portion 500 of FIG. 5A and the ultrasound device 100. Those components are not described in detail here. The portion 600 comprises a heat sink 602 disposed within the opening of interposer 504. The heat sink 602 is formed of a thermally conductive material and is provided instead of the thermal vias 216 of FIG. 3. The heat sink 602 has a thermal conductivity equal to any of those listed previously herein with respect to the thermal vias 216. The heat sink 602 may be formed of copper in some embodiments. The heat sink 602 contacts the heat spreader 502 and the shroud adapter 604. The shroud adapter 604 may be substantially the same as the shroud adapter 506 of FIG. 5A, but may differ in shape to accommodate the heat sink 602. The heat sink 602 may be mounted to the interposer 504 using surface mount technology (SMT). Heat may be conducted from the ultrasound-on-chip device 202 to the shroud adapter 604 and the housing 102 via the heat sink 602 in the direction indicated by arrow 606.

An ultrasound device including the construction shown in FIG. 6 may include the additional thermal dissipation features illustrated in FIG. 2. For example, a housing with a single piece construction and a chassis supporting a circuit board may be provided.

As described above, ultrasound devices such as ultrasound probes of the types described herein may include an ultrasound-on-chip device. A non-limiting example of an ultrasound-on-chip device which may be implemented in the various embodiments described herein including ultrasonic transducers and integrated circuitry is illustrated in schematic form in FIG. 7. The ultrasound-on-chip device 700 may include one or more transducer arrays 702, a transmit (TX) control circuit 704, a receive (RX) circuit 706, a timing and control circuit 708, a signal conditioning/processing circuit 710, and/or a power management circuit 718 receiving ground (GND) and voltage reference (V_{IN}) signals. Optionally, a high intensity focused ultrasound (HIFU) controller (not shown) may be included if the ultrasound-on-chip device is to be used to provide HIFU. In the embodiment shown, all of the illustrated elements are formed on a single semiconductor die (or substrate or chip) 712. Alternatives are possible, though. For instance, the transducer array 702 may be formed on one chip and the illustrated circuitry on one or more additional chips that are bonded to the chip with the transducers. In addition, although the illustrated example shows both a TX control circuit 704 and an RX circuit 706, in alternative embodiments only a TX control circuit or only an RX control circuit may be employed. For example, such embodiments may be employed in a circumstance in which the ultrasound-on-chip device is operated as a transmission-only device to transmit acoustic signals or a reception-only device used to receive acoustic signals that have been transmitted through or reflected by a subject being ultrasonically imaged, respectively.

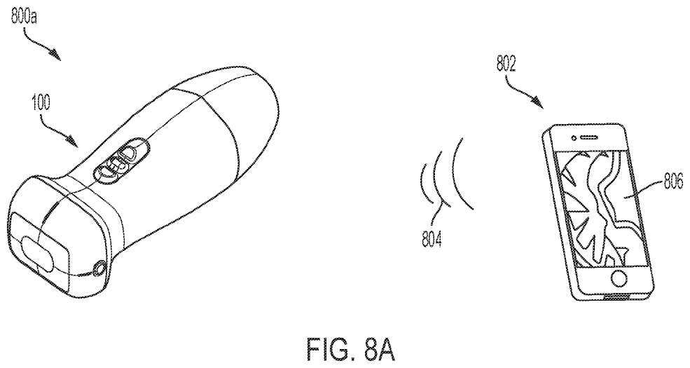

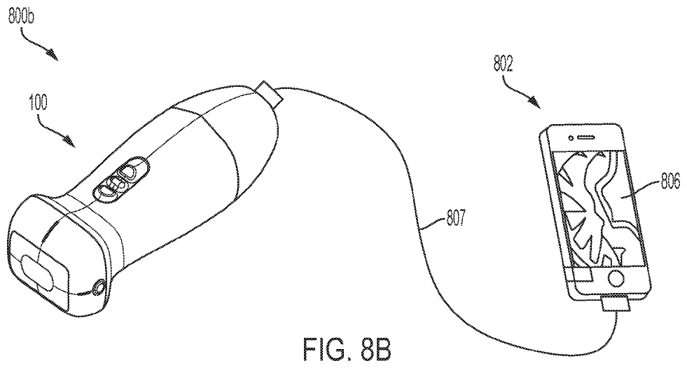

The ultrasound-on-chip device 700 further includes a serial output port 714 which may represent an implementation of an interface for communicating data off the semiconductor die. The serial output port 714 may be a wireless port or a wired connection in some embodiments. Both types of connections are shown below in FIGS. 8A and 8B, respectively.

The ultrasound-on-chip device 700 may also include a clock input port 716 to receive and provide a clock signal CLK to the timing and control circuit 708.

The transducer array 702 may include various numbers of transducers and the transducers may be of various types. As described previously herein, the ultrasonic transducers may be microscale ultrasonic transducers. In some embodiments, the ultrasonic transducers are CMUTs or PMUTs. The ultrasonic transducers may number in the hundreds or thousands (e.g., between 5,000-10,000). The ultrasonic transducers may be arranged in a 1D, 1.5D, or 2D array.

The RX circuit 706 may include various components to process signals from the ultrasonic transducers. In some embodiments, a TIA is provided for each ultrasonic transducer, or for substantially each ultrasonic transducer. Thus, in some embodiments several thousand TIAs are provided. As an example, the transducer array 702 may include approximately 8,000 ultrasonic transducers, and the RX circuit 706 may include 8,000 respective TIAs. Additionally, analog-to-digital converters (ADCs) may be provided in the RX circuit 706. For example, approximately 1,000 ADCs may be provided to digitize signals provided by the approximately 8,000 TIAs. In some embodiments, one ADC is provided for each 6-12 ultrasonic transducers. Such a large number of TIAs and ADCs contributes meaningfully to the heat generation of the ultrasound-on-chip device.

Ultrasound-on-chip devices for use in the types of ultrasound devices described herein may include alternative or additional circuitry to that shown in FIG. 7. The circuitry may be analog circuitry, digital circuitry, or a combination.

As described previously, operation of the circuitry and the ultrasonic transducers generates heat. The amount of heat generated depends on the power consumption of the circuitry and the duration of the circuitry operation. In some embodiments, the power consumption of the circuitry of the ultrasound-on-chip device may be multiple Watts. That, in combination with the power consumption of any additional circuitry of the ultrasound device, such as the circuitry of circuit boards 212 and 214, may amount to several Watts. For example, the total power consumption of the ultrasound device (e.g., ultrasound device 100) may be at least 3 Watts, at least 4 Watts, at least 5 Watts, between 3 and 10 Watts, or any value within that range. Improved ultrasound imaging performance is often achieved at higher power consumption levels since the circuitry can generate stronger ultrasound signals and sometimes process signals more quickly at higher power consumption rates. Thus, providing for higher power consumption during operation of the device can be preferable as described previously herein.

Ultrasound devices of the types described herein may communicate with external processing devices. The external processing devices may send control signals to and/or receive output data from the ultrasound device. FIGS. 8A and 8B illustrate examples. Referring to FIG. 8A, the ultrasound system 800a includes the ultrasound device 100 and a processing device 802. The processing device is configured to communicate wirelessly with the ultrasound device 100 with wireless signals 804. The processing device 802 may send wireless signals 804 to the ultrasound device 100 and receive wireless signals from the ultrasound device 100. The processing device 802 includes a display screen 806 for displaying data, instructions, and/or ultrasound images.

The processing device 802 may take various forms. In the non-limiting example of FIG. 8A, the processing device 802 is a smartphone. However, alternative devices may be used, such as a tablet computer or a laptop computer.

FIG. 8B illustrates an alternative to the system 800a. In the alternative embodiment of FIG. 8B, the ultrasound system 800b is configured to communicate with the processing device 802 via a wired connection 807. The wired connection 807 may, for example, provide a connection to serial output port 714 of FIG. 7. The wired connection 807 may be a universal serial bus (USB) connection, or other suitable connection. In at least some embodiments, the wired connection 807 may be of a type used for consumer electronics. In at least some embodiments, the ultrasound system may be operable as both a wireless system and a wired system. That is, FIGS. 8A and 8B may represent different modes of operation of the same ultrasound system in some embodiments.

Having thus described several aspects and embodiments of the technology of this disclosure, it is to be appreciated that various alterations, modifications, and improvements will readily occur to those of ordinary skill in the art. For example, the ultrasound device 100 is illustrated as an ultrasound probe, but alternatively may be a wearable ultrasound patch. Such alterations, modifications, and improvements are intended to be within the spirit and scope of the technology described in the disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Elements other than those specifically identified by the “and/or” clause may optionally be present, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

What is claimed is:

1. An apparatus, comprising:
a handheld ultrasound probe weighing between 100 grams and 500 grams, having a length of less than 300 mm, and being couplable to a smartphone or tablet, the handheld ultrasound probe containing:
a handheld housing;
a rear cap coupled to the handheld housing;
a shroud coupled to the handheld housing;
a lens coupled to the shroud, wherein the handheld housing, rear cap, shroud, and lens are coupled to define an enclosed space;
a semiconductor chip or chip stack disposed behind the lens within the enclosed space and comprising an array of microscale ultrasonic transducers and integrated circuitry;
an interposer disposed behind the semiconductor chip or chip stack within the enclosed space; and
at least one circuit board disposed behind the interposer within the enclosed space and electrically coupled to the interposer,
wherein the interposer comprises a plurality of epoxy-filled copper-plated thermal vias having inner diameters between 5 mil and 15 mil, spaced from each other at a pitch between 10 mil and 30 mil, and covering in combination between 5% and 15% of an area of one side of the interposer, and
wherein the handheld ultrasound probe is configured to operate in a runtime mode with a power consumption of at least 5 Watts for at least 15 minutes.

2. The apparatus of claim 1, wherein the handheld housing is formed of a material having a thermal conductivity coefficient of at least 50 (W m−1 K−1) at 20° C. and 1 bar, and wherein the handheld housing has a single piece construction.

3. The apparatus of claim 1, further comprising a chassis disposed within the handheld housing and coupled to the at least one circuit board.

4. The apparatus of claim 1, further comprising a shroud adapter positioned between the interposer and the handheld housing, the shroud adapter having a thermal conductivity coefficient of at least 50 (W m−1 K−1) at 20° C. and 1 bar.

5. The apparatus of claim 4, wherein the plurality of epoxy-filled copper-plated thermal vias of the interposer contact the shroud adapter.

6. The apparatus of claim 1, further comprising:
a chassis disposed within the handheld housing and coupled to the at least one circuit board; and
a shroud adapter positioned between the interposer and the handheld housing, the shroud adapter having a thermal conductivity coefficient of at least 50 (W m−1 K−1) at 20° C. and 1 bar,
wherein the handheld housing is formed of a material having a thermal conductivity coefficient of at least 50 (W m−1 K−1) at 20° C. and 1 bar and has a single piece construction.

7. The apparatus of claim 1, wherein the plurality of epoxy-filled copper-plated thermal vias comprises between 400 and 500 epoxy-filled copper-plated thermal vias.

8. The apparatus of claim 1, wherein the integrated circuitry of the semiconductor chip or chip stack includes between 80 and 120 nets, and wherein the interposer comprises a pin connector having between 100 and 200 pins.

9. The apparatus of claim 1, wherein the handheld ultrasound probe is wirelessly operatively couplable to the smartphone or tablet.

10. The apparatus of claim 1, wherein the plurality of epoxy-filled copper-plated thermal vias are spread over an area between 80 mm² and 150 mm².

11. A method of operating an ultrasound apparatus, comprising:
with a semiconductor chip or chip stack comprising an array of microscale ultrasonic transducers and integrated circuitry, transmitting and receiving ultrasound signals through a lens of the ultrasound apparatus; and
dissipating heat generated by the semiconductor chip or chip stack through a plurality of epoxy-filled copper-plated thermal vias disposed in an interposer coupled to a back of the semiconductor chip or chip stack, the plurality of epoxy-filled copper-plated thermal vias covering in combination between 5% and 15% of an area of one side of the interposer.

12. The method of operating the ultrasound apparatus of claim 11, further comprising conducting heat from the plurality of epoxy-filled copper-plated thermal vias to a housing of the ultrasound apparatus, the housing having a thermal conductivity coefficient of at least 50 (W m−1 K−1) at 20° C. and 1 bar.

13. The method of operating the ultrasound apparatus of claim 12, wherein conducting the heat from the plurality of epoxy-filled copper-plated thermal vias to the housing of the ultrasound apparatus comprises conducting the heat through a shroud adapter of the ultrasound apparatus, the shroud adapter having a thermal conductivity coefficient of at least 50 (W m−1 K−1) at 20° C. and 1 bar.

14. The method of operating the ultrasound apparatus of claim 12, further comprising conducting the heat along a length of the housing.

15. The method of operating the ultrasound apparatus of claim 11, further comprising absorbing heat generated by a circuit board of the ultrasound apparatus using a chassis to which the circuit board is mounted.

16. An ultrasound imaging apparatus, comprising:
a semiconductor chip or chip stack comprising an array of microscale ultrasonic transducers and integrated circuitry; and
an interposer coupled to the semiconductor chip or chip stack and comprising a plurality of dedicated thermal vias covering between 5% and 15% of an area of a side of the interposer and configured to dissipate heat generated by the semiconductor chip or chip stack.

17. The ultrasound imaging apparatus of claim 16, wherein the microscale ultrasonic transducers are capacitive micromachined ultrasonic transducers (CMUTs).

18. The ultrasound imaging apparatus of claim 16, wherein the plurality of dedicated thermal vias are thermally connected on the side of the interposer to a conductive sheet region.

19. The ultrasound imaging apparatus of claim 16, further comprising:
a heat spreader disposed between the semiconductor chip or chip stack and the interposer; and
a heat sink,
wherein the interposer comprises an opening and wherein a portion of the heat sink extends through the opening and makes contact with the heat spreader.

20. The ultrasound imaging apparatus of claim 16, further comprising:
a heat spreader disposed between the semiconductor chip or chip stack and the interposer;
a heat sink; and
a shroud adapter having a thermal conductivity coefficient of at least 50 (W m−1 K−1) at 20° C. and 1 bar,
wherein the heat sink is disposed between the heat spreader and the shroud adapter.
